(19) United States
(12) Patent Application Publication
(10) Pub. No.: US 2011/0281744 A1
(43) Pub. Date: Nov. 17, 2011

(54) METHOD OF IDENTIFYING COMPOUNDS THAT MODULATE REGULATION OF IRON RESPONSE ELEMENTS

(76) Inventors: Anthony Giordano, Shreveport, LA (US); Abner Mhashilkar, Rockville, MD (US)

(21) Appl. No.: 12/599,866

(22) PCT Filed: May 14, 2008
(86) PCT No.: PCT/US08/06146

§ 371 Date: May 16, 2011

Related U.S. Application Data
(60) Provisional application No. 60/930,111, filed on May 14, 2007.

(51) Int. Cl. C40B 30/04; C12Q 1/68

(52) U.S. Cl. 506/9; 435/6.19

(57) ABSTRACT

The invention features methods for identifying compounds that inhibit binding between iron response elements (IREs) and iron response proteins (IRPs). These compounds are potentially useful for treating or preventing diseases, in particular cancer, but also including anemia, neurodegenerative diseases, inflammation and iron overload. The methods are based on contacting a candidate compound in an in vitro system and monitoring the binding of an IRE to an IRP. In addition, the invention features methods of treating or preventing a proliferative disease, such as cancer, using the compounds of the invention.

### CROSS-REFERENCE TO RELATED APPLICATIONS

This application claims benefit of U.S. Provisional Application No. 60/930,111, filed 14 May 2007, which is hereby incorporated by reference.

### BACKGROUND OF THE INVENTION

The invention relates to the fields of screening assays and detection of compounds and methods for treating cancer and other diseases. More specifically, this invention describes high throughput screening methods for altering iron homeostasis at the post-transcriptional level.

Iron homeostasis is well recognized to be critical in cell survival, with either too much or too little iron resulting in cellular death. It is also known that iron is critical for cell proliferation (Robbins, PNAS, 1970; Neckers, Cancer Investigation, 1986) likely through its interaction with ribonucleotide reductase, the rate limiting enzyme required for DNA synthesis (Thelander, Ann Rev. Biochem., 1979; Atkin, J B C, 1973). In addition, one of the key proteins involved in iron homeostasis, the transferrin receptor (TfR) is found at higher levels in cancer cells (Gatter, J. Clin. Pathol., 1983; Rudland, BBRC, 1977). In fact, a common imaging agent used to observed tumors in patients, gallium, is taken up by the TfR, distinguishing the cancer cells containing high levels of receptors from normal cells (Weiner, Nucl. Med. Biol., 1996). More recently, the overexpression of TfR in cancer cells has led to development of cytotoxic monoclonal antibodies for the treatment of cancer (reviewed in Daniels, Clin Immunol., 2006).

While TfR is responsible for transporting transferring bound iron into the cell, other proteins also play key roles in iron homeostasis. Another protein, the divalent metal transporter 1 (DMT1) is responsible for transporting iron across the endosomal membrane to be released in the cytosol. Once in the cytosol, a third protein, ferritin, can complex and sequester the free iron. Interestingly, the regulation of iron homeostasis occurs predominantly at the post-transcriptional level (reviewed in Thomson, Int. J. Biochem Cell Biol., 1999). This post-transcriptional regulation is the result of an RNA binding protein binding to similar cis-elements in the 5′ or 3′ UTRs of the target mRNAs.

The post-transcriptional regulation of the RNA binding protein with a cis-element, called an iron response element (IRE) was first reported in the 5′ UTR of the ferritin mRNA and was demonstrated to inhibit ferritin protein synthesis (Hentze, PNAS, 1987). Soon after, a similar IRE was found in the 3′ UTR of the TfR, and when bound by the RNA binding protein the TfR mRNA was stabilized and protein levels increased (Rouault, Science, 1988). The RNA binding protein, now called Iron Response Protein 1 (IRP 1) was identified shortly thereafter (Rouault, 1990, PNAS). Thus, ferritin protein synthesis is inhibited under conditions of low iron, through this post-transcriptional interaction of IRP 1 with the IRE found in the 5′ UTR of the ferritin mRNA, while the same binding to the IREs in the 3′ UTR of TfR leads to increased protein levels. Conversely, when iron levels increase, IRP1 no longer binds to the IREs, destabilizing TfR mRNA and reducing TfR protein levels (thereby reducing iron uptake) and releases the translational repression of ferritin, increasing ferritin levels (resulting in increasing storage of free iron). A second IRP (IRP2) was cloned (Guo, J B C, 1994) and it has been shown that IRP1 and IRP2 bind to distinct sets of mRNAs (Henderson, J B C, 1996). The function of the two IRPs is reviewed in Rouault (Nat Chem Biol, 2006) and Pantopoulos (Ann Ny Acad Sci, 2004). A similar IRE is also found in the 3′ UTR of two of the four isoforms of DMT1 (Hubert, PNAS, 2002). Like TfR, binding of the IRP to the IRE of DMT1 leads to mRNA stabilization and increased protein expression. The DMT1 isoforms do not appear to be functionally different, rather they are localized to different regions of the cell (Mackenzie, Biochem J., 2007).

As mentioned above, tumor cells require high levels of iron for proliferation. Thus, strategies for reducing iron levels have been advanced as therapies for treatment of cancer. Iron chelators are the most advanced compounds being studied for reducing iron levels in cancer patients. Desferrioxamine is widely used as a treatment for iron overload disease with another iron chelator, Triapine, in Phase II clinical trials. A recent screen of potential chelators in cellular proliferation and clonogenic assays resulted in the identification of novel chelators. Importantly, these molecules inhibited growth of tumors in a xenograft model without systemic effects, which was attributed to the low dosages needed for antitumor activity (Whitnall, PNAS, 2006). Curcumin, which is in trials as an anti-cancer agent, also acts as an iron chelator.

The above studies indicate the utility of identifying molecules that inhibit iron in cancer cells as a therapy. However, curcumin treatment also leads to increased IRP activation, which in turn leads to an increase in TfR and a decrease in ferritin (Brodie, Cancer Res., 1993; Jiao, Free Radic Biol Med., 2006). Hence, it appears that as iron levels are reduced by chelation, the cancer cells attempt to compensate by post-transcriptional regulation of the IRP/IRE mRNAs. Another approach to lowering intracellular iron in cancer cells is to reduce the levels of TfR or DMT1 proteins by preventing IRP binding to the IRE.

Sodium ascorbate may in fact exhibit anti-tumor activity through this mechanism. In melanoma cells, sodium ascorbate treatment causes a reduction in TfR, which in turn leads to reduced intracellular iron and eventually apoptosis (Kang, J. Cell. Physiol., 2005). Likewise, a naturally occurring peptide, hepcidin, reduces DMT1 protein isoforms containing the IRE, but not those lacking the IRE. This reduction in IRE(+) DMT1 mRNA is sufficient to significantly reduce iron uptake (Yamaji, Blood, 2004). These studies further demonstrate the utility of reducing iron transport by altering the post-transcriptional regulation of the IRP with the IRE. Screening for small molecules acting at the post-transcriptional level have been reported (Ecker, Drug Disc Today, 1999; Xavier, Trends Biotech., 2000) but have been very limited for identifying small molecules inhibiting IRE/IRP interactions. A cellular reporter gene assay using the IRE from APP was carried out (Venti, Ann NY Acad Sci, 2004) and led to the identification of a number of metal chelators. More recently, a chemical foot-printing screen of small molecules directly effecting IRP binding to the ferritin mRNA IRE led to the identification of yohimbine (Tibodeau, PNAS, 2006). Since this screen requires the use of gel electrophoresis to distinguish the chemical footprints, it is extremely low throughput.

The present invention describes in high throughput vitro screens capable of identifying compounds acting at the post-transcriptional level to alter IRP/IRE interactions. Such in vitro screens have the advantage over cellular screens in that no iron chelators will be identified and the advantage over the chemical footprinting screen in that the assays are high throughput. In addition, by counter-screening, with IREs from other mRNA, specificity of the compound can be determined. The use of highly specific or general IRE modulators may have different utility in treating various cancers or diseases.

### SUMMARY OF THE INVENTION

The invention features screening assays for compounds that are potentially useful for treating or preventing a proliferative disease, such as cancer, and other diseases, such as inflammation, anemia, neurodegenerative disease and iron overload. Additionally, the invention features methods of treating or preventing a proliferative disease using the compounds of the invention.

In one aspect the invention features a method of identifying a candidate compound that modulates post-transcriptional regulation of one or more IRE-containing mRNAs. This method including measuring the binding of the one or more IRE-containing mRNAs to IRP in the presence and absence of a test compound. A difference in binding indicates the test compound is a candidate compound for modulating the post-transcriptional regulation of the one or more IRE-containing mRNAs.

In the forgoing aspect of the invention, the one or more IRE-containing mRNAs is labeled (e.g., fluorescently labeled, and the measuring is fluorescent polarization) and introduced to a well, the IRP is immobilized to the well, and the measuring includes the measuring of the labeled one or more IRE-containing mRNAs in the well.

Alternatively, the IRP is labeled and introduced to a well, the one or more IRE-containing mRNAs is immobilized to the well, and the measuring includes the detection of the labeled IRP in the well.

In another aspect, the one or more IRE-containing mRNAs is fluorescently labeled, the IRP is labeled with a quencher, and the measuring includes measuring the level of fluorescence. Alternatively, the IRP is fluorescently labeled, the one or more IRE-containing mRNAs are labeled with a quencher, and the measuring includes measuring the level of fluorescence.

In any of the forgoing aspect, the one or more (e.g., 2, 3, 4, or more) IRE-containing mRNAs can be selected from IREs found in transferring (e.g., SEQ ID NOs: 3-7), ferritin (e.g., SEQ ID NOs: 1 and 2), or DMT1 (e.g., SEQ ID NO: 8), or from other IRE sequences (e.g., those set forth in FIG. 5) and the IRP can be selected from IRP1 and IRP2.

Further, in any of the forgoing methods, the label can be selected from a fluorescent or radioactive label and the difference in binding can be at least 25%.

In another aspect, the invention features the additional step of contacting a cell with the candidate compound and measuring at least one disease-associated property of the cell in the presence and absence of the candidate compound. In this aspect, the cell is a model for an iron uptake related disorder (e.g., cancer, anemia, inflammation, neurodegenerative disease and iron overload disease), and a decrease in the one or more disease-associated properties identifies the candidate compound as a candidate compound for treating the iron uptake associated disorder.

In another aspect, the invention features methods for treating or preventing a proliferative disease, such as cancer, in a subject, e.g., a mammal or human. The methods include administering to the subject a therapeutically effective amount of a chemical compound identified in the screens. The compound may be in a pharmaceutically acceptable carrier. The therapeutically effective amount is, for example, a dosage sufficient to modulate IRE/IRP interaction, altering iron uptake, resulting in toxicity or growth arrest to cancer cells, without inducing general systemic toxicity.

By “candidate compound” or “compound” is meant a chemical, be it naturally-occurring or artificially-derived, that is screened by employing one of the assay methods described herein. Candidate compounds may include, for example, peptides, polypeptides, synthetic organic molecules, naturally occurring organic molecules, nucleic acid molecules, sugars, polysaccharides, and derivatives thereof.

By “iron uptake related disorder” is meant to include proliferative diseases, for example, prostate cancer, breast cancer, gastrointestinal cancer, lung cancer, colon cancer, melanoma, ovarian cancer, gastric cancer, bladder cancer, salivary gland carcinoma, a brain tumor, leukemia, lymphoma, carcinoma, and the symptoms associated with cancer. This term is also meant to include myeloproliferative and degenerative diseases (such as autoimmune disorders-Parkinson's, Alzheimer's, RA, Neuropathological disabilites/disorders) anemia, inflammation, neurodegenerative diseases and iron overload diseases.

By a “dosage sufficient to modulate IRE/IRP interaction” is meant an amount of a chemical compound or small molecule that increases or decreases the interaction of an IRP with the targeted IRE when administered to a subject. For example, for a compound that decreases TfR IRE interaction with an IRP, the modulation leads to a in TfR mRNA stability and a corresponding decrease in TfR protein expression that is at least 10%, 30%, 40%, 50%, 75%, or 90% lower in a treated subject than in the same subject prior to the administration of the inhibitor or than in an untreated, control subject. In addition, for a compound that increases TfR IRE interaction with an IRP, the amount of UR mRNA and protein, for example, is at least 1.5-, 2-, 3-, 5-, 10-, or 20-fold greater in a treated subject than in the same subject prior to the administration of the modulator or than in an untreated, control subject.

By “modulate” is meant a change in the level of IRE/IRP interaction, as measured by a change in the level of detected label, as assayed, for example, by time-resolved fluorescence. The change is, for example, at least 1.5-fold to 2-fold, by at least 3-fold to 5-fold, or by at least 10-fold to 20-fold, relative to a control sample that was not administered the compound, or that was contacted with the compound vehicle only.

By “IRE” is meant one or more of the cis-acting elements obtained from TfR mRNA, ferritin mRNA, of DMT1 mRNA that interacts with an IRP. For example, the sequences that correspond to sequences substantially identical to the sequences set forth in TfR (e.g., SEQ ID NOs: 3-7), ferritin (e.g., SEQ ID NOs: 1 and 2), or DMT1 (e.g., SEQ ID NO: 8), or from other IRE sequences (e.g., those set forth in FIG. 5).

By “substantially identical” is meant a nucleic acid sequence that, when optimally aligned, for example using the methods described below, share at least 75%, 80%, 85%, 90%, 95%, 96%, 97%, 98%, 99%, or 100% sequence identity with a second nucleic acid. Percent identity between two nucleic acid sequences is determined in various ways that are within the skill in the art, for instance, using publicly available computer software such as Smith Waterman Alignment (Smith, T. F. and M. S. Waterman (1981) J. Mol. Biol. 147:195-7); “Best Fit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool; (Altschul, S. F., W. Gish, et al. (1990) J. Mol. Biol. 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. It is understood that for the purposes of determining sequence identity when comparing a DNA sequence to an RNA sequence, a thymine nucleotide is equivalent to a uracil nucleotide.

By “IRP” is meant the iron responsive protein capable of interacting with an IRE. The protein can be IRP-1, IRP-2 or both.

By “label” is meant any molecule, whether fluorescent, radioactive or other, that can be detected. For example, a fluorescent label may be detected by use of a fluoremeter and a radioactive label by a scintillation counter.

By “modulates” is meant changing the level of IRE binding to the IRP, either by decrease or increase.

By “proliferative disease” is meant a disease that is caused by or results in inappropriately high levels of cell division, inappropriately low levels of apoptosis, or both. For example, cancers such as lymphoma, leukemia, melanoma, ovarian cancer, breast cancer, pancreatic cancer, bladder cancer, gastric cancer, salivary gland carcinoma, and lung cancer are all examples of proliferative disease. A myeloproliferative disease is another example of a proliferative disease.

By “promoter” is meant a minimal sequence sufficient to direct transcription.

By “protein” or “polypeptide” or “polypeptide fragment” is meant any chain of more than two amino acids, regardless of post-translational modification (e.g., glycosylation or phosphorylation), constituting all or part of a naturally-occurring polypeptide or peptide, or constituting a non-naturally occurring polypeptide or peptide.

By “regulatory element” is meant sequences that can modulate expression of a gene or gene product. Examples of regulatory sequences include, but are not limited to promoters, enhancers, sequences that stabilize an RNA sequence, sequences that enhance protein stability, translation termination sequences, and additional 5′ or 3′ UTR sequences.

By “therapeutically effective amount” is meant an amount of a compound sufficient to produce a preventative, healing, curative, stabilizing, or ameliorative effect in the treatment of a condition, e.g., a proliferative disease.

By “treating” is meant the medical management of a subject, e.g. an animal or human, with the intent that a prevention, cure, stabilization, or amelioration of the symptoms or condition will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder. “Treating” a condition with the compounds of the invention involves administering such a compound, alone or in combination and by any appropriate means, to an animal, cell, lysate or extract derived from a cell, or a molecule derived from a cell.

### BRIEF DESCRIPTION OF THE DRAWINGS

FIG. 1A top panel is a graph showing polysome analysis with optical density as a function of percent sucrose. Total RNA is isolated from cultured cells by lysing the cells in polysome extraction buffer (15 mM Tris, pH 7.6, 0.3 M NaCl, 15 mM MgCl2 and 1% Triton X-100) containing 100 ng/ml cyclohexamide and 1 mg/ml heparin. The lysates are then layered onto a 10-50% sucrose gradient and centrifuged at 38,000 rpm for 2 hrs. The gradients are fractionated by monitoring UV absorbance at 254 nm.

FIG. 1A bottom panel is a northern blot showing Actin and Ferratin RNA. RNA was isolated from each fraction using RNeasy 96 Universal Tissue Kit (Qiagen) then loaded on a agarose gel and subjected to northern analysis with the appropriate probe. The high molecular weight fractions (6-12) represent those for which protein synthesis is occurring.

FIG. 1B are northern blots showing the indicated RNA. Cells were treated overnight with either iron oriron plus desferal to chelate iron and RNA was isolated and analyzed as described above. As can be seen from the panel on the left, neither iron nor deseferal treatment had an effect on the translation of actin mRNA. Conversely, from the panel on the right, it can be seen that when iron is added, ferritin translation increases while sequestration of iron with desferal inhibits translation.

This is consistent with the IRP binding the ferritin IRE in low iron and inhibiting protein synthesis while addition of iron prevents binding and increases ferritin synthesis.

FIG. 1C left panel is an autoradiogram showing the rate of synthesis of the indicated protein. Cells were treated as above, with 35S-methionine added 1 hour before isolation of protein. Ferritin was then immunoprecipitated with antibodies and the amount of newly synthesized protein was determined to confirm the polysomes.

FIG. 1C right panel is a graph showing percentage of Ferratin synthesis under each of the indicated conditions.

FIG. 2 is the ferritin IRE sequence and corresponding Genbank accession numbers.

FIG. 3 is the transferring (TfR) IRE sequence and corresponding Genbank accession numbers.

FIG. 4 is the DMT1 IRE sequence and corresponding Genbank accession numbers.

FIG. 5 are additional IRE sequences and corresponding Genbank accession numbers.

### DETAILED DESCRIPTION OF THE INVENTION

Assays for identifying compounds for use in treating or preventing a proliferative disease, e.g., by modulating the level of intracellular iron through post-transcriptionally altering the interaction of an iron response element (IRE) with an IRP (iron response protein), are described herein. These assays involve identifying compounds which modulate binding between an IRE and an IRP. Preferably these in vitro assays are carried out in a high throughput fashion. Following the identification of candidate compounds, the compounds are further analyzed in cellular assays to determine whether the compounds can reduce a disease associated characteristic. The identified compounds can potentially be used in the treatment of proliferative diseases, such as various cancers.

### IRE Molecules

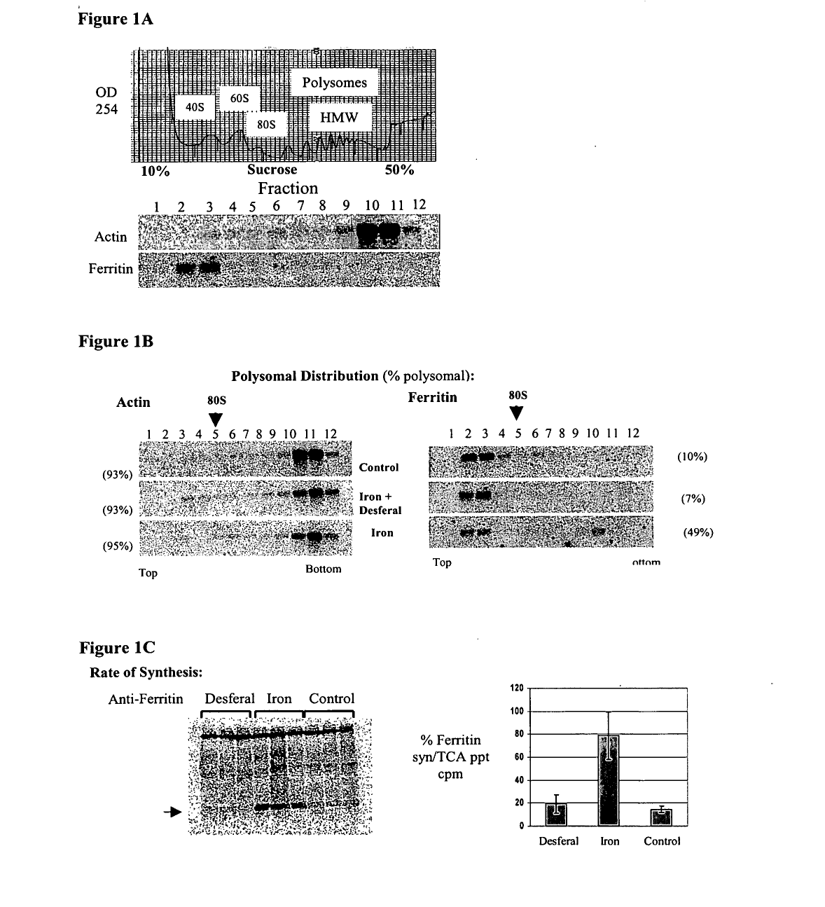

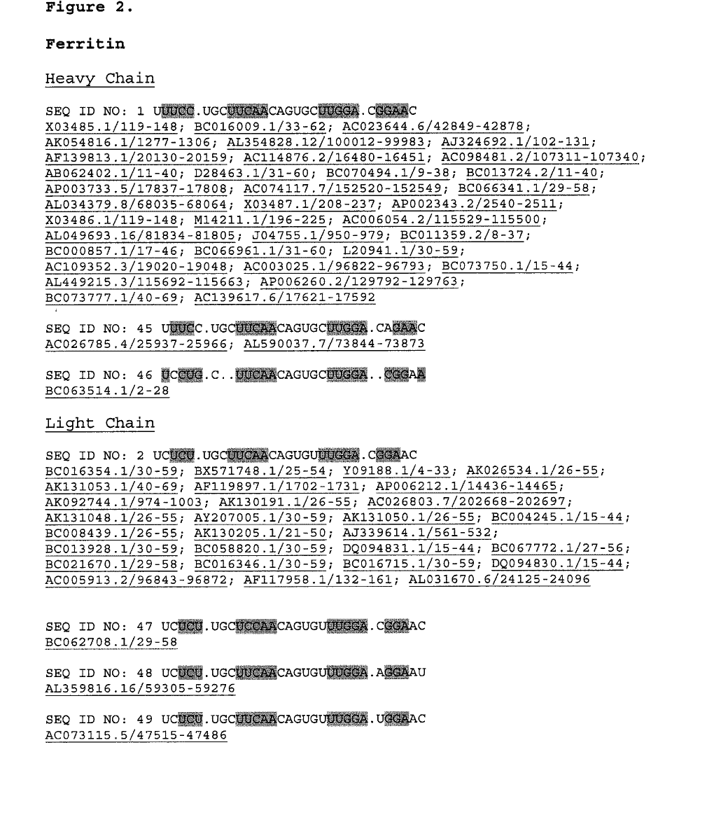

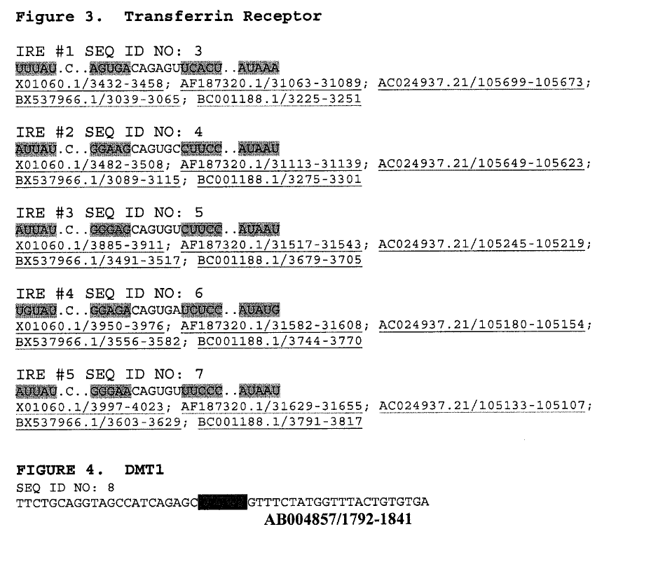

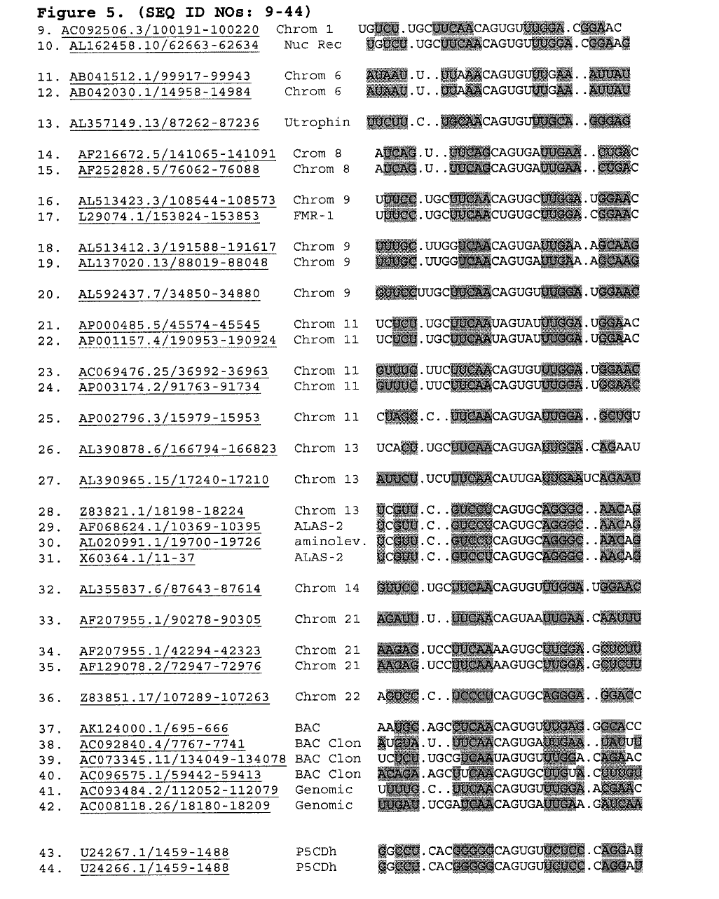

IRE molecules were identified by polysome analysis (FIGS. 1A-1C). Exemplary IRE sequences are set forth in FIGS. 2-5. IRE RNA molecules can be made by in vitro transcription or by direct synthesis. IRE RNA molecules can be used either in isolation or in combination with one or more other IRE RNA molecules, selected from the set of IREs found in TfR, ferritin, and DMT1. Preferably, one type of IRE RNA molecule is used, and most preferably, the IRE from TfR is used in the primary screen. Subsequent screens for specificity of a compounds activity will utilize all three of the IREs.

IRE RNA molecules can be produced recombinantly using known techniques, (e.g., by in vitro transcription and by direct synthesis). For recombinant and in vitro transcription, DNA encoding RNA molecules can be obtained from clones known in the art, by synthesizing a DNA molecule encoding an RNA molecule, or by cloning the gene encoding the IRE RNA molecules. Techniques for in vitro transcription of RNA molecules and methods for cloning genes encoding known IRE RNA molecules are described by, for example, Sambrook et al.

Detection of interactions between the IRE and IRP molecules can be facilitated by attaching a detectable label to the IRE molecule. Generally, labels known to be useful for nucleic acids can be used to label the IRE RNA molecules. Examples of suitable labels include radioactive isotopes (e.g., ³³P, ³²P, and ³⁵S), fluorescent labels (e.g., fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7), biotin, and europium.

Labeled nucleotides are the preferred form of label since they can be directly incorporated into the RNA molecules during synthesis. Examples of detection labels that can be incorporated into amplified RNA include nucleotide analogs such as BrdUrd (Hoy and Schimke, Mutation Research 290:217-230 (1993)), BrUTP (Wansick et al., J. Cell Biology 122:283-293 (1993)), nucleotides modified with biotin (Langer et al., Proc. Natl. Acad. Sci. USA 78:6633 (1981)), and suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res. 22:3226-3232 (1994)). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Labels that are incorporated into RNA molecules, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1.sup.3,7]decane]-4-yl) phenyl phosphate; Tropix, Inc.).

The IRE RNA molecules can be individually labeled with the same label and individually screened. It is also possible to label all three IREs with different labels that do not interfere with each other and screen all IREs in a single well.

### IRP Molecules

IRP-1 and IRP-2 can be purified from normal cells or tissue by methods such as those described by Henderson, JBC, 1993, using commercially available antibodies, such as those sold by Santa Cruz, or antibodies generated against IRP-1 and/or IRP-2 using standard antibody generation methods. Additionally, the DNA encoding the proteins can be cloned into a vector, transfected, expressed and purified from a high expressing system such as Pichia pastoris (Allerson, RNA, 2003). Recombinant IRP proteins can also be produced by standard methods in bacteria, yeast or mammalian cells. The recombinant IRP can also be produced in an in vitro translation system through addition of IRP mRNA, which can be produced recombinantly from plasmid DNA in an in vitro transcription system. Rabbit reticulocytes and wheat germ extracts are two such systems in which IRP can be produced. The transcription and translation can also be coupled in a single system. Recombinant IRP proteins are further purified using antibodies specific to the IRP and standard molecular techniques, such as immobilizing the antibody on an affinity column or immunoprecipitation.

Recombinant IRP proteins can be labeled directly using fluorescent, radioactive, biotin or other labels as appropriate using techniques or tool kits, such as the Alexa Fluor Kit from Invitrogen. Recombinant proteins can also be labeled during their synthesis through the incorporation of labeled amino acids, such as ³⁵S-methionine, FluoroTecT Green Lysine (Promega), biotinylated lysine or other appropriately labeled amino acids.

Screeening for Compounds that Modulate IRE/IRP Interactions

The invention features screening assays (e.g., high throughput screening assays) for identification of specific or broadly acting IRE/IRP modulators. Previous screening strategies have made use of cellular assays incorporating a related IRE from APP into a reporter gene or low through-put footprinting assays. The cellular assay led to the identification of iron chelators, which have been demonstrated by others to actually increase TfR levels and reduce ferritin levels, presumably as a compensatory mechanism in response to low cellular iron. For this reason, the invention features a direct in vitro screen. Screens of the invention include four main components: the IRE, the IRP, a binding buffer and the compound(s) to be tested.

High throughput, in vitro screens can be either carried out, for example, with the IRE or IRP attached to a solid support, such as the well of a plate or in solution. Attaching the IRP to a well of a screening plate can be achieved through hydrophobic interactions of the protein with the plastic, by first attaching an antibody against the IRP to an IgG coated plate then adding the IRP or, alternatively, if the protein has been labeled with biotin, streptavidin coated plates can be used. The IRE RNA is most easily attached to the plate using a biotin at one end of the molecule. The addition of a tether, consisting of a string of non-specific nucleotides or carbons, for example, may be used to improve binding activity.

In the screen, either IRP or IRE will be immobilized to a well of a screening plate. Binding buffer will be added to each well, followed by addition of IRE or IRP and optionally a test compound. The remaining IRE/IRP component and compound can be added simultaneously, or in any order. Examples of appropriate binding buffers can be found in U.S. Pat. No. 6,107,029 and in many of the articles referenced herein. If cellular extracts are used instead of IRP, an additional non-specific competitor, such as poly(G), heparin or tRNA may be added to reduce non-specific binding. The components are then incubated at 20° C. to 37° C. for between 5 minutes and 4 hours. A washing may be required prior to detection, for example, when the non-bound IRE/IRP is labeled with fluorescence or radioactivity. In other cases the washing step could be eliminated, for example, when a radioactive proximity assay is carried out. Detection can occur by directing analyzing the label or indirectly, for example using streptavidin-complexed with horse radish peroxidase when biotin is used as a label. In addition, detection can be carried out by ELISA screening when the IRP is not attached to the plate. Typical immobilized high throughput screens include fluorescent assays, time-resolve fluorescence assays, absorption assays, ELISAs, radioactive proximity assays, radioactive binding assays, and other assays routinely used to monitor a compounds effect on a macromolecular interaction.

Solution based assays can also be used to identify compounds capable of modulating the IRE/IRP interaction. Such solution based assays include FRET assays when both the IRE and IRP are labeled, where energy transfer from one of the components is monitored. Alternatively, the invention features fluorescent polarization assays. Here, the IRE would be labeled with a fluorescent tag which upon binding to the much larger IRP, would result in a change in the polarization of the signal.

In many high throughput assay formats, the assay can be multiplexed so that more than one interaction can be detected in a single well. For example, all three IREs could be labeled with a different, non-interfering fluorescent label. Similarly, each of the IRPs could be individually tagged with a different label. Thus, the high throughput assay can be carried out with an individual IRE and IRP or combinations of IREs and IRPs.

### In Vivo Assays

The invention features the optional step of testing a compound identified in the above described in vitro screens for desirably activity in a cell culture model of a disease. The use of cell based reporter gene assays can provide additional information concerning the ability of a compound to enter a cell and modulate intracellular IRE/IRP interaction. Such methods are described, for example, in U.S. Pat. No. 7,078,171. Briefly, the IRE from TfR, ferritin, and DMT1 are separately cloned 5′ (ferritin) or 3′ (TfR and DMT1) to a reporter gene (e.g., luciferase, CAT, or green fluorescent protein) under the control of a ubiquitous or inducible transcriptional promoter. Some examples of promoters commonly used are CMV, EF-1alpha, RSV and SV40. Also, while the isolated IRE element can be used, it is preferred to clone the entire 5′ and 3′ UTRs from TfR, ferritin, and DMT1 in the proper position and orientation relative to the reporter gene. Following transfection (e.g., transiently or stably transfected cells), candidate compounds can be tested in a high throughput fashing for the ability to alter the post-transcriptional regulation of the targeted mRNA intracellularly.

The above cellular assay can be carried out in any cell line although it is most preferred to carryout the assay in a cell line that endogenously expresses the IRE and IRP of interest. If the IRP is not expressed, cells may also be transfected with a expression plasmid with the IRP of interest.

### Analysis of Post—Trancriptional Actvity

The invention also features the optional step of confirming the ability of a candidate compound to effect the post-transcriptional regulation of the targeted IRE/IRP interactions and determining the precise cellular mechanism of action. Since both TfR and DMT1 mRNA are stabilized following binding of the IRP to the IRE, mRNA stability can be monitored. The stability of mRNA can be determined in transfected cells by first blocking transcription with a compound such as actinomycin D (5.mu.g/ml), and then measuring the degradation rate of the transcripts by quantifying their level in cells harvested at different times. To quantify the level of transcript, total cell RNA purified from harvested cells is subjected to electrophoresis followed by transfer to a filter by pressure blotting. Following incubation, the filter is subject to hybridization by a radiolabeled probe designed to detect the transcript sequence. Additionally, real time quantitative PCR with total cell RNA can be used for quantitating mRNA degradation rates. Such degradation rates are calculated, for example, by densitometric scanning of the autoradiographs (Saulnier-Blache et al., Mol. Pharmacol. 50: 1432 42, 1996; Yang et al., J. Biol. Chem. 272: 15466 15473, 1997). A decrease in the rate of degradation indicates an increase in mRNA stability. A compound that was found to increase In addition to monitoring TfR and DMT1, ferritin and actin stability can be monitored as a control, since these would not be expected to change following compound treatment.

Ferritin is regulated at the level of protein synthesis with IRP binding to its IRE repressing translation. Thus, a small molecule that inhibits this binding should result in increased ferritin synthesis whereas a small molecule that increases binding of the IRP to the ferritin IRE should result in decreased protein synthesis. A number of strategies exist to monitor protein synthesis, which can be used to analyze a compounds effectiveness at this level. One common strategy is to monitor polysome distribution of the RNA of interest. Following treatment of the appropriate cell line with and without the candidate compound, cells are lysed and RNA is isolated. The RNA is layered onto a sucrose gradient and following ultracentrifugation, fractions are collected. The fractions are then assayed for distribution of the desired mRNA by northern analysis or rtPCR as described in US Patent Application 20030170702. Compounds that inhibit the IRP interaction with the ferritin IRE should result in a shift from the low molecular weight polysomes found near the top of the gradient to high molecular weight polysomes found near the bottom of the gradient. Another well established means of analyzing a specific change in protein synthesis id to pulse the cells with a labeled amino acid then immunoprecipitate the protein of interest. After treatment with or without the candidate compounds, the cells are starved with an amino acid free media then incubated with labeled amino acid for a short period of time 5-60 minutes. Cells are then lysed and protein is immunoprecipitated from each sample with the appropriate antibody.

### Toxicity and Growth Inhibitory Assays

In colorectal cancers there is an increase in both DMT1 and TfR (Brookes, Gut, 2006); the IRE-containing DMT1 was increased in gliobastoma cells (L is, Mol. Brain. Res., 2004); and TfR is increased in pancreatic cancers (Ryschich, Eur J Cancer, 2004), suggesting that either or both the down regulation of TfR and DMT1 through their IRE/IRP interaction may represent effective strategies for the treatment of cancer. However, in other studies there was weak to no TfR protein expression in highly metastatic tumors (Prutki, Cancer Lett, 2006) and an apparent TfR5-independent iron uptake in AML cells (Nakamaki, Br J. Haematol., 2004). Thus, it is unclear how alteration of a specific protein involved in iron homeostasis might effect tumor cell growth and likely that modulation of different proteins will be important for treating different cancers. For example, a DMT1 inhibitor may be much more effective for treating TfR-independent tumor cells and provide a better safety profile than a broad IRP/IRE inhibitor, whereas in colorectal cancers a broad inhibitor may be the ideal strategy for use as a treatment. A critical aspect of this invention will be to analyze the effects of candidate compounds on cancer cell growth and viability.

The viability of a cell, either from a primary tumor or a cell line representing a primary tumor, contacted with a candidate compound may be used to screen for compounds that are useful for treating or preventing proliferative diseases. Reduced iron uptake in tumor cells has been shown to be associated with cell toxicity. Therefore, a compound that decreases the interaction of an IRE/IRP will result in decreased expression of TfR or DMT1 or an increase in ferritin, each of which will lead to a decrease in intracellular iron and should lead to increased cell death or inhibit cell proliferation, compared to control cells that are not administered the compound, or that are contacted with the compound vehicle only. In addition, an assay based on viability of cells will detect compounds whose toxicity is mediated by another mechanism. Methods for assaying cell death are well known in the art. For example, cell death can be measured by determining cellular ATP levels, wherein a cell that is undergoing cell death has a decreased level of cellular ATP compared to a control cell. Cell death may also be measured by staining with a vital dye, for example, trypan blue, wherein a cell that is dying will be stained with the vital dye, and a cell that is not dying will not be stained with the dye. Fluorescent dyes can also be used, with detection using equipment such as that sold by Cellomics. Additionally, kits are available from companies such as Promega for detecting changes in proliferation or toxicity.

In some instances, inhibition of cell growth may be sufficient for a therapeutic effect. As such, assays for cell viability may also be used to determine if candidate compounds inhibit cell growth. Inhibition can be measured, for example by determining by standard means the number of cells in a population contacted with the candidate compound compared to the number of cells in a population not contacted with the candidate compound. If the number of cells in the population contacted with the candidate compound does not increase over time or increases at a reduced rate compared to cells not contacted with the compound, the candidate compound inhibits the growth of the cells. A similar assay may also be performed to determine if a compound stimulates growth.

### Therapy

A compound identified by any of the above-described methods may be administered within a pharmaceutically acceptable diluent, carrier, or excipient, in unit dosage form. Conventional pharmaceutical practice may be employed to provide suitable formulations or compositions to administer the identified compound to patients suffering from a proliferative disease. Administration may begin before the patient is symptomatic. Any appropriate route of administration may be employed, for example, administration may be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” (19th ed., A. R. Gennaro, ed., 1995, Mack Publishing Company, Easton, Pa.). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds that modulate HER2 translation efficiency include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a compound identified according to the methods described above, may be combined with more traditional therapies for a proliferative disease, for example, traditional chemotherapeutic agents, radiation therapy, or surgery. In addition, these methods may be used to treat any subject, including mammals, for example, humans, domestic pets, or livestock.

The criteria for assessing response to therapeutic modalities employing an identified compound is dictated by the specific condition and will generally follow standard medical practices. Generally, the effectiveness of administration of the compound can be assessed by measuring changes in characteristics of the disease condition.

### REFERENCES

Atkin, C. L., Thelander, L., Reichard, P., Lang, G. (1973) Iron and free radical in ribonucleotide reductase. Exchange of iron and Moosbauer spectroscopy of the protein B2 subunit of the Escherichia coli enzyme. J. Biol. Chem. 248: 7464-7472.
Daniels, T R, Delgado, T., Rodrguez, J. A., Helguera, G., Penichet, M. L. (2006) The transferrin receptor part I: Biology and targeting with cytotoxic antibodies for the treatment of cancer. Clin Immunol. 121:144-58.
Ecker, D. J., Griffey, R. H. (1999) RNA as a small-molecule drug target: doubling the value of genomics.Drug Disc Today 4: 420-429.
Gatter, K. C., Brown, G., Trowbridge, I. S., Woolston, R. E., Mason, D. Y. (1983) Transferrin receptors in human tissues: their distribution and possible clinical relevance. J. Clin. Pathol., 36: 539-545.
Gua, B., Yu, Y., Leibold, E. A. (1994) Iron regulates cytoplasmic levels of a novel iron-responsive element-binding protein without aconitase activity. J. Biol. Chem. 269:24252-24260.
Henderson, B. R., Menotti, E., Kuhn, L. C. (1996) Iron regulatory proteins 1 and 2 bind to distinct sets of RNA targets. J. Biol. Chem. 271: 4900-4908.
Hentze, M. W., Rouault, T. A., Caughman, S. W., Dancis, A., Harford, J. B., Klausner, R. D. (1987) A cis-acting element is necessary and sufficient for translational regulation of human ferritin expression in response to iron. PNAS 84: 6730-6734.
Hubert, N., Hentze, M. W. (2002) Previously uncharacterized isoforms of divalent metal transporter (DMT)-1: Implications for regulation and cellular function. PNAS 99: 12345-12350.
Jiao, Y., Wilkinson, J. 4^{th}, Christine Pietsch, E., Buss, J. L., Wang, W., Planalp, R., Torti, F. M., Torti, S. V. (2006) Iron chelation in the biological activity of curcumin. Free Radic Biol Med. 40: 1152-1160.
Kang, J. S., Cho, D., Kim, Y. I., Hahm, E., Kim, Y. S., Jin, S. N., Kim, H. N., Kim, D., Hur, D., Park, H., Hwang, Y. I., Lee, W. J. (2005) Sodium ascorbate (vitamin C) induces apoptosis in melanoma cells via the down-regulation of transferrin receptor dependent iron uptake. J. Cell Physiol. 204: 192-197.
Mackenzie, B., Takanaga, H., Hubert, N., Rolfs, A., Hediger, M. A. (2007) Functional properties of multiple isoforms of human divalent metal-ion transporter 1 (DMT1). Biochem J. 403:59-69.
Neckers, L. M., Trepel, J. B. (1986) Transferrin receptor expression and the control of cell growth. Cancer Investig. 4: 461-470.
Pantopoulos, K. (2004) Iron metabolism and the IRE/IRP regulatory system: an update. Ann NY Acad. Sci. 1012:1-13.
Robbins, E., Pederson, T. (1970) Iron: Intracellular localization and possible role in cell division. PNAS, 66: 1244-1251.
Rouault, T. A., Hentze, M. W., Caughman, S. W., Harford, J. B., Klausner, R. D. (1988) Binding of a cytosolic protein to the iron-responsive element of human ferritin messenger RNA. Science 241: 1207-1210.
Rouault, T. A., Tang, C. K., Kaptain, S., Burgess, W. H., Haile, D. J., Klausner, R. D. (1990) Cloning of the cDNA encoding an RNA regulatory protein—the human iron-responsive element-binding protein. PNAS 87: 7958-7962.
Rouault, T. A. (2006) The role of iron regulatory proteins in mammalian iron homeostasis and disease. Nat Chem. Biol. 2: 406-414.
Rudland, P. S., Durbin, H., Clingan, D., deAusa, L. J. (1977) Iron salts and transferrin are specifically required for cell division of cultured 3T6 cells. Biochem Biophys Res Comm. 75: 556-562.
Thelander, L., Reichard, D. R. (1979) Reduction of ribonucleotides. Ann Rev. Biochem. 48:133-158.
Tibodeau, J. D., Fox, P. M., Ropp, P. A., Theil, E. C., Thorp, H. H. (2006) The up-regulation of ferritin expression using a small-molecule ligand to the native mRNA. (2006) PNAS103: 253-257.
Thomson, A. M.; Rogers, J. T.; Leedman, P. J. (1999) Iron-regulatory proteins, iron-responsive elements and ferritin mRNA translation. Int. J. Biochem Cell Biol. 31:1139-52.
Weiner, R. E. (1996) The mechanism of ⁶⁷Ga localization in malignant disease. Nucl. Med. Biol. 23:745-751.
Whitnall, M., Howard, J., Ponka, P., Richardson, D. R. (2006) A class of iron chelators with a wide spectrum of potent antitumor activity that overcomes resistance to chemotherapeutics. PNAS103:14901-14906.
Venti, A., Giordano, T., Eder, P., Bush, A. I., Lahiri, D. K., Greig, N. H., Rogers, J. T. (2004) The integrated role of desferrioxamine and phenserine targeted to an iron-response element in the APP-mRNA 5′-untranslated region. Ann NY Acad. Sci. 1035:34-48.
Xavier, K. A., Eder, P. S., Giordano, T. (2000) RNA as a drug target: methods for biophysical characterization and screening. Trends Biotechnol. 18: 349-356.
Yamaji, S., Sharp, P., Ramesh, B., Srai, S. K. (2004) Inhibition of iron transport across human intestinal epithelial cells by hepcidin. Blood 104: 2178-2180.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of identifying a candidate compound that modulates post-transcriptional regulation of one or more IRE-containing mRNAs, said method comprising measuring the binding of said one or more IRE-containing mRNAs to IRP in the presence and absence of a test compound; wherein a difference in binding indicates said test compound is a candidate compound for modulating said post-transcriptional regulation of said one or more IRE-containing mRNAs.

2. The method of claim 1, wherein said one or more IRE-containing mRNAs is labeled and introduced to a well, wherein said IRP is immobilized to said well, and wherein said measuring comprises the measuring of said labeled one or more IRE-containing mRNAs in said well.

3. The method of claim 2, wherein said one or more IRE-containing mRNAs is fluorescently labeled, and said measuring is fluorescent polarization.

4. The method of claim 1, wherein said IRP is labeled and introduced to a well and said one or more IRE-containing mRNAs is immobilized to said well, and wherein said measuring comprises the detection of said labeled IRP in said well.

5. The method of claim 1, wherein said one or more IRE-containing mRNAs is fluorescently labeled, wherein said IRP is labeled with a quencher, and wherein said measuring comprises measuring the level of fluorescence.

6. The method of claim 1, wherein said IRP is fluorescently labeled, wherein said one or more IRE-containing mRNAs are labeled with a quencher, and wherein said measuring comprises measuring the level of fluorescence.

7. The method of claim 1, wherein said one or more IRE-containing mRNAs is selected from transferrin receptor IRE, ferritin IRE, and DMT1 IRE.

8. The method of claim 7, wherein said method comprises measuring the binding of two or more said IRE-containing mRNAs to IRP in the presence and absence of a test compound, wherein said two or more said IRE-containing mRNAs are selected from transferrin receptor IRE, ferritin IRE, and DMT1 IRE.

9. The method of claim 7, wherein said method comprises measuring the binding of three of said IRE-containing mRNAs to IRP in the presence and absence of a test compound, wherein said three or more said IRE-containing mRNAs are transferrin receptor IRE, ferritin IRE, and DMT1 IRE.

10. The method of claim 1, wherein said IRP is selected from IRP1 and IRP2.

11. The method of claim 2, wherein said label is selected from a fluorescent or radioactive label.

12. The method of claim 1 wherein said difference in binding is at least 25%.

13. The method of claim 1, wherein said method comprises a high throughput screen.

14. The method of claim 1, further comprising contacting a cell with said candidate compound and measuring at least one disease-associated property of said cell in the presence and absence of said candidate compound, wherein said cell is a model for an iron uptake related disorder, and wherein a decrease in said one or more disease-associated properties identifies said candidate compound as a candidate compound for treating said iron uptake associated disorder.

15. The method of claim 14, wherein said iron uptake associated disorder is selected from the group consisting of cancer, anemia, inflammation, neurodegenerative disease and iron overload disease.


SEQUENCE LISTING (49 sequences)

SEQ ID NO: 1 - RNA, 32 nt, Artificial Sequence
  Synthetic oligonucleotide
uuuccnugcuucaacagugcuuggancggaac

SEQ ID NO: 2 - RNA, 32 nt, Artificial Sequence
  Synthetic oligonucleotide
ucucunugcuucaacaguguuuggancggaac

SEQ ID NO: 3 - RNA, 32 nt, Artificial Sequence
  Synthetic oligonucleotide
uuuauncnnagugacagaguucacunnauaaa

SEQ ID NO: 4 - RNA, 32 nt, Artificial Sequence
  Synthetic oligonucleotide
auuauncnnggaagcagugccuuccnnauaau

SEQ ID NO: 5 - RNA, 32 nt, Artificial Sequence
  Synthetic oligonucleotide
auuauncnngggagcagugucuuccnnauaau

SEQ ID NO: 6 - RNA, 32 nt, Artificial Sequence
  Synthetic oligonucleotide
uguauncnnggagacagugaucuccnnauaug

SEQ ID NO: 7 - RNA, 32 nt, Artificial Sequence
  Synthetic oligonucleotide
auuauncnngggaacaguguuucccnnauaau

SEQ ID NO: 8 - DNA, 50 bp, Artificial Sequence
  Synthetic oligonucleotide
ttctgcaggtagccatcagagccagugugtttctatggtttactgtgtga

SEQ ID NO: 9 - RNA, 32 nt, Artificial Sequence
  Synthetic oligonucleotide
ugucunugcuucaacaguguuuggancggaac

SEQ ID NO: 10 - RNA, 32 nt, Artificial Sequence
  Synthetic oligonucleotide
ugucunugcuucaacaguguuuggancggaag

SEQ ID NO: 11 - RNA, 32 nt, Artificial Sequence
  Synthetic oligonucleotide
auaaununnuuaaacaguguuugaannauuau

SEQ ID NO: 12 - RNA, 32 nt, Artificial Sequence; identical to SEQ ID NO: 11
  Synthetic oligonucleotide

SEQ ID NO: 13 - RNA, 32 nt, Artificial Sequence
  Synthetic oligonucleotide
uucuuncnnugcaacaguguuugcanngggag

SEQ ID NO: 14 - RNA, 32 nt, Artificial Sequence
  Synthetic oligonucleotide
aucagnunnuucagcagugauugaanncugac

SEQ ID NO: 15 - RNA, 32 nt, Artificial Sequence; identical to SEQ ID NO: 14
  Synthetic oligonucleotide

SEQ ID NO: 16 - RNA, 32 nt, Artificial Sequence
  Synthetic oligonucleotide
uuuccnugcuucaacagugcuugganuggaac

SEQ ID NO: 17 - RNA, 32 nt, Artificial Sequence
  Synthetic oligonucleotide
uuuccnugcuucaacugugcuuggancggaac

SEQ ID NO: 18 - RNA, 32 nt, Artificial Sequence
  Synthetic oligonucleotide
uuugcnuuggucaacagugauugaanagcaag

SEQ ID NO: 19 - RNA, 32 nt, Artificial Sequence; identical to SEQ ID NO: 18
  Synthetic oligonucleotide

SEQ ID NO: 20 - RNA, 32 nt, Artificial Sequence
  Synthetic oligonucleotide
guuccuugcuucaacaguguuugganuggaac

SEQ ID NO: 21 - RNA, 32 nt, Artificial Sequence
  Synthetic oligonucleotide
ucucunugcuucaauaguauuugganuggaac

SEQ ID NO: 22 - RNA, 32 nt, Artificial Sequence; identical to SEQ ID NO: 21
  Synthetic oligonucleotide

SEQ ID NO: 23 - RNA, 32 nt, Artificial Sequence
  Synthetic oligonucleotide
guuucnuucuucaacaguguuugganuggaac

SEQ ID NO: 24 - RNA, 32 nt, Artificial Sequence; identical to SEQ ID NO: 23
  Synthetic oligonucleotide

SEQ ID NO: 25 - RNA, 32 nt, Artificial Sequence
  Synthetic oligonucleotide
cuagcncnnuucaacagugauugganngcugu

SEQ ID NO: 26 - RNA, 32 nt, Artificial Sequence
  Synthetic oligonucleotide
ucacunugcuucaacagugauuggancagaau

SEQ ID NO: 27 - RNA, 32 nt, Artificial Sequence
  Synthetic oligonucleotide
auucunucuuucaacauugauugaaucagaau

SEQ ID NO: 28 - RNA, 32 nt, Artificial Sequence
  Synthetic oligonucleotide
ucguuncnnguccucagugcagggcnnaacag

SEQ ID NO: 29 - RNA, 32 nt, Artificial Sequence; identical to SEQ ID NO: 28
  Synthetic oligonucleotide

SEQ ID NO: 30 - RNA, 32 nt, Artificial Sequence; identical to SEQ ID NO: 28
  Synthetic oligonucleotide

SEQ ID NO: 31 - RNA, 32 nt, Artificial Sequence; identical to SEQ ID NO: 28
  Synthetic oligonucleotide

SEQ ID NO: 32 - RNA, 32 nt, Artificial Sequence
  Synthetic oligonucleotide
guuccnugcuucaacaguguuugganuggaac

SEQ ID NO: 33 - RNA, 32 nt, Artificial Sequence
  Synthetic oligonucleotide
agauununnuucaacaguaauugaancaauuu

SEQ ID NO: 34 - RNA, 32 nt, Artificial Sequence
  Synthetic oligonucleotide
aagagnuccuucaaaagugcuuggangcucuu

SEQ ID NO: 35 - RNA, 32 nt, Artificial Sequence; identical to SEQ ID NO: 34
  Synthetic oligonucleotide

SEQ ID NO: 36 - RNA, 32 nt, Artificial Sequence
  Synthetic oligonucleotide
aguccncnnucccucagugcagggannggacc

SEQ ID NO: 37 - RNA, 32 nt, Artificial Sequence
  Synthetic oligonucleotide
aaugcnagccucaacaguguuugagnggcacc

SEQ ID NO: 38 - RNA, 32 nt, Artificial Sequence
  Synthetic oligonucleotide
auguanunnuucaacagugauugaannuauuu

SEQ ID NO: 39 - RNA, 32 nt, Artificial Sequence
  Synthetic oligonucleotide
ucucunugcgucaauaguguuuggancagaac

SEQ ID NO: 40 - RNA, 32 nt, Artificial Sequence
  Synthetic oligonucleotide
acaganagcuucaacagugcuuguancuuugu

SEQ ID NO: 41 - RNA, 32 nt, Artificial Sequence
  Synthetic oligonucleotide
uuuugncnnuucaacaguguuugganacgaac

SEQ ID NO: 42 - RNA, 32 nt, Artificial Sequence
  Synthetic oligonucleotide
uugaunucgaucaacagugauugaangaucaa

SEQ ID NO: 43 - RNA, 32 nt, Artificial Sequence
  Synthetic oligonucleotide
ggccuncacgggggcaguguucuccncaggau

SEQ ID NO: 44 - RNA, 32 nt, Artificial Sequence; identical to SEQ ID NO: 43
  Synthetic oligonucleotide

SEQ ID NO: 45 - RNA, 32 nt, Artificial Sequence
  Synthetic oligonucleotide
uuuccnugcuucaacagugcuuggancagaac

SEQ ID NO: 46 - RNA, 32 nt, Artificial Sequence
  Synthetic oligonucleotide
uccugncnnuucaacagugcuugganncggaa

SEQ ID NO: 47 - RNA, 32 nt, Artificial Sequence
  Synthetic oligonucleotide
ucucunugcuccaacaguguuuggancggaac

SEQ ID NO: 48 - RNA, 32 nt, Artificial Sequence
  Synthetic oligonucleotide
ucucunugcuucaacaguguuugganaggaau

SEQ ID NO: 49 - RNA, 32 nt, Artificial Sequence
  Synthetic oligonucleotide
ucucunugcuucaacaguguuugganuggaac